(19) United States
(12) Patent Application Publication
(10) Pub. No.: US 2021/0393323 A1
(43) Pub. Date: Dec. 23, 2021

(54) ELECTRODE SUCTION DRILL SYSTEM

(71) Applicant: IMEDICOM CO., LTD., Gunpo-si, Gyeonggi-do (KR)

(72) Inventor: Don Soo JU, Gunpo-si, Gyeonggi-do (KR)

(73) Assignee: IMEDICOM CO., LTD., Gunpo-si, Gyeonggi-do (KR)

(21) Appl. No.: 17/291,576

(22) PCT Filed: Oct. 18, 2019
(86) PCT No.: PCT/KR2019/013729

§ 371 Date: May 5, 2021

(30) Foreign Application Priority Data
Nov. 9, 2018 (KR) 10-2018-0137336

(51) Int. Cl. A61B 18/14; A61B 34/00

(52) U.S. Cl. CPC A61B 18/1492; A61B 2018/00339; A61B 34/71

(57) ABSTRACT

The present disclosure relates to an electrode suction drill system including a manipulator and a hollow catheter tube, and including: a first electrode and a second electrode provided apart from each other on a front end of the catheter tube; a steering ring provided at an inner front end of the catheter tube; first and second wires positioned inside the catheter tube and having one ends connected to the steering ring; a drill wire provided in an inner hollow portion of the catheter tube; a thread formed on an outer circumferential surface of the drill wire; and a motor provided inside the manipulator and fastened to one end of the drill wire to rotate the drill wire, in which a tip of the catheter tube performs a steering operation by a manipulation of the first and second wires, and as the drill wire is rotated by the rotation of the motor, lesion site tissue in a body is suctioned into the catheter tube by the thread.

### CROSS-REFERENCE TO RELATED APPLICATIONS

This application claims priority from Korean Patent Application No. 10-2018-0137336, filed on Nov. 9, 2018 in the Korean Intellectual Property Office, the invention of which is incorporated herein by reference in its entirety.

### BACKGROUND

### Technical field

The present disclosure relates to a technology for performing decompression more effectively for the treatment of a patient diagnosed with the lumbar herniated intervertebral discs, by inserting a catheter into a patient in his diagonal direction, and performing decompression treatment using an electrode, while removing the tissues from the lesion site.

### Background Art

The lumbar herniated intervertebral disc is a disease that causes the back pain and neurological symptoms due to bulging intervertebral disc. As the intervertebral disc (between spinal columns) undergoes aging, circumferential fissure and radial tear occur in the fibrous ring surrounding the edge of the intervertebral disc, and in particular, the lumbar herniated intervertebral disc can be caused when the patient is excessively bending to lift a heavy object, or the like, as the nucleus pulposus at the center of the intervertebral disc is subject to the pressure force and torsional power, and cannot remain in stack in the fibrous ring and escape out of radial cracks.

As a way of treatment for this, a method is proposed, which includes inserting a radiation frequency electrode body into the internal tissue of the intervertebral disc, and removing the tissue around the electrode body into a gaseous state by separating the constituent materials of the nucleus pulposus into negatively charged electrons and positively charged ions with a high-energy electric field formed using the radiation frequency. Such technology is disclosed in Korean Patent Application Publication No. 10-2011-0098698, for example. The radiation frequency has a frequency range of 100 to 20,000 kHz, and the radiation frequency electrode is used to excise or remove the body tissue and discard waste products or the like from the blood vessels, and because the radiation frequency electrode body approaches further inside of the body through an inner diameter of a needle after the needle is inserted into a region to be treated, the electrode body must be implemented to be smaller than the inner diameter of the guiding needle.

The general related radiation frequency electrode body has a straight shape, and thus has a limited insertion position and accessible site in the body, and, for the treatment of the lumbar herniated intervertebral discs, in particular, due to the presence of the spine and the nerves of the spine, the location where the tip of the radiation frequency electrode can be inserted is very limited, and accordingly, it is very difficult to remove the tissue portion that is opposite the insertion position of the radiation frequency electrode.

In addition, while the related technology is to insert a catheter inside the disc and perform treatment by using the electrode, there is a problem that the sufficient effect of decompression cannot be anticipated, because there is no structure provided to remove it when a certain region is burned due to the temperature, or to discharge removed parts from the lesion tissue inside the body or substances such as gelatin and the like within the disc to outside.

### DETAILED DESCRIPTION

### Technical Problem

An object of the present disclosure is to provide a device for treating lumbar herniated intervertebral disc, with a technology capable of more effectively decompressing inside the disc.

### Technical Solution

There is provided an electrode suction drill system including a manipulator 100 and a hollow catheter tube 200 mounted on the manipulator, which may include a first electrode and a second electrode provided apart from each other on a front end of the catheter tube, a steering ring 230 provided at an inner front end of the catheter tube, first and second wires 231 and 232 positioned inside the catheter tube and having one ends connected to the steering ring, a drill wire 250 provided in an inner hollow portion of the catheter tube, a thread 253 formed on an outer circumferential surface of the drill wire, and a motor M provided inside the manipulator and fastened to one end of the drill wire to rotate the drill wire, in which a tip of the catheter tube performs a steering operation by a manipulation of the first and second wires, and as the drill wire is rotated, lesion site tissue in a body is suctioned into the catheter tube by the thread.

The thread includes a tip-side thread 253 formed at a foremost end of the drill wire, and one or more spaced apart threads 255 formed on the outer circumferential surface of the drill wire.

An embodiment further includes a motor position adjusting unit 140 positioned on an upper surface of the manipulator to move the motor in a front-and-rear direction, and a coupling part 170 to fixedly couple the catheter tube inside the manipulator, in which, when the motor is moved in the front-and-rear direction by the motor position adjusting unit, only the drill wire is relatively movable in the front-and-rear direction while the catheter tube remains fixed to the manipulator.

The manipulator further includes a circular rotating plate to pull the first wire or the second wire to thus pull the steering ring, such that the tip of the catheter tube performs the steering operation, and an angle adjusting unit 130 formed integrally with the circular rotating plate to rotate the circular rotating plate.

In addition, an embodiment may further include a discharge member 300 provided inside the manipulator and into which an end of the catheter tube is inserted, a discharge passage 310 provided inside the manipulator, in which one end of the discharge passage is connected with the discharge member, and a suction port 320 provided outside the manipulator and connected to the other end of the discharge passage to discharge the lesion site tissue to outside.

### Technical Effects

With the configuration described above according to the present disclosure, the catheter is used in the treatment of the lumbar herniated intervertebral discs and decompression can be performed more effectively, since the lesion tissue, gelatin within the disc, or the like can be removed concurrently while the treatment using the electrode is performed.

### BRIEF DESCRIPTION OF THE DRAWING

FIG. 1 is an overall schematic diagram of an electrode suction drill system according to an embodiment of the present disclosure.

FIG. 2 is a view of a catheter tube of an electrode suction drill system according to the present disclosure.

FIG. 3 is a cross-sectional view of a catheter tube of an electrode suction drill system according to the present disclosure.

FIG. 4 is a view of a drill wire of an electrode suction drill system according to the present disclosure.

FIG. 5 is an enlarged view of a portion of a manipulator of an electrode suction drill system according to the present disclosure.

FIGS. 6 and 7 is a view of a catheter tube of an electrode suction drill system in operation according to the present disclosure.

FIG. 8 is a view of a drill wire advancing and protruding from a catheter tube of an electrode suction drill system according to the present disclosure.

### BEST MODE

The objectives, specific advantages and novel features of the present invention will become more apparent from the following detailed description and the preferred embodiments, which are associated with the accompanying drawings. In addition, terms described herein are terms defined in consideration of functions in the present invention, which may vary according to the intention or convention of a user or an operator. Therefore, definitions of these terms should be made based on the contents throughout the present specification.

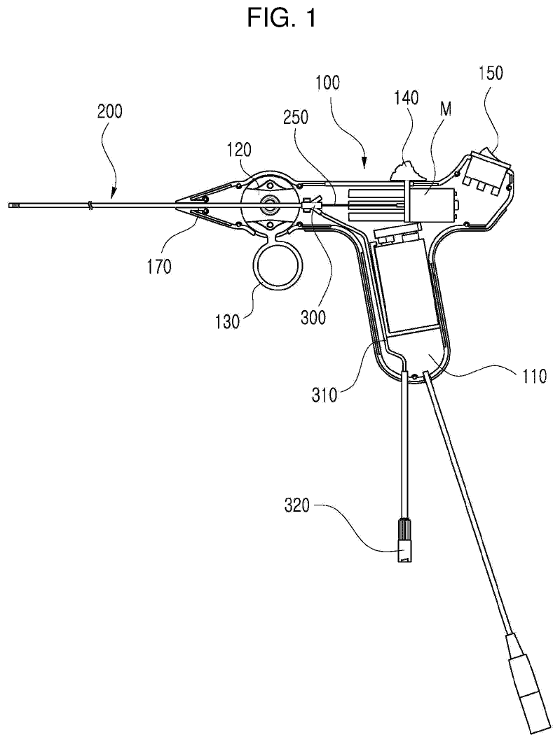

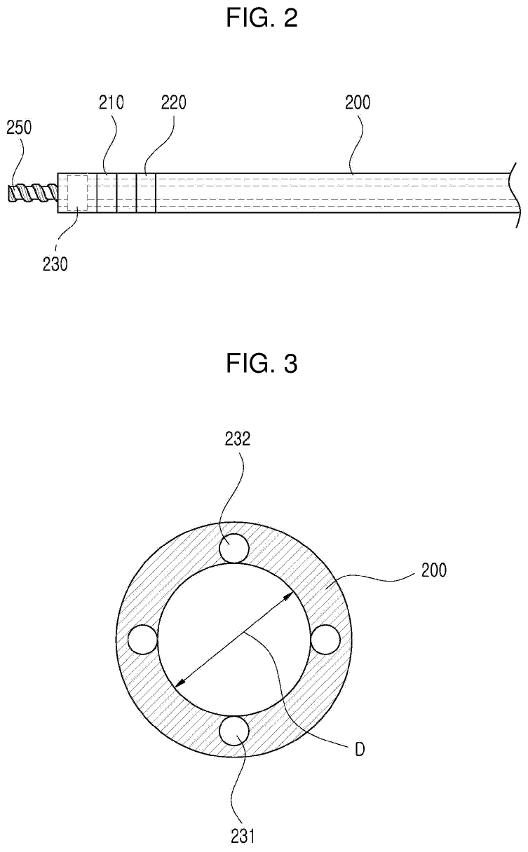

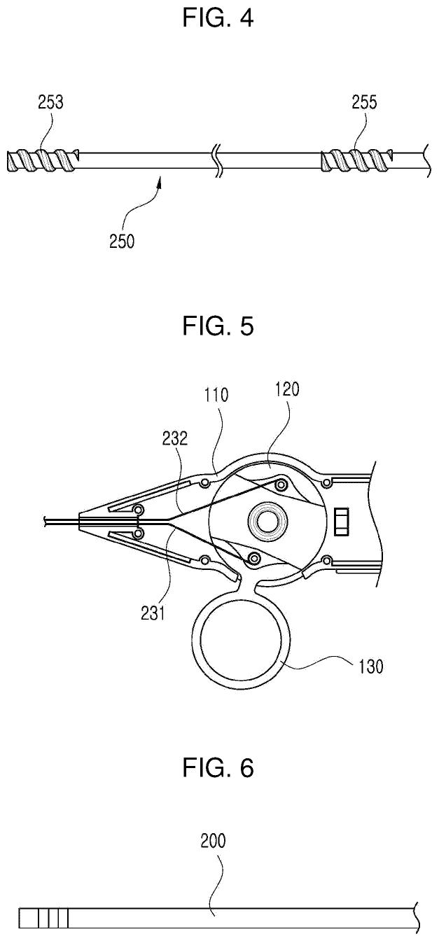

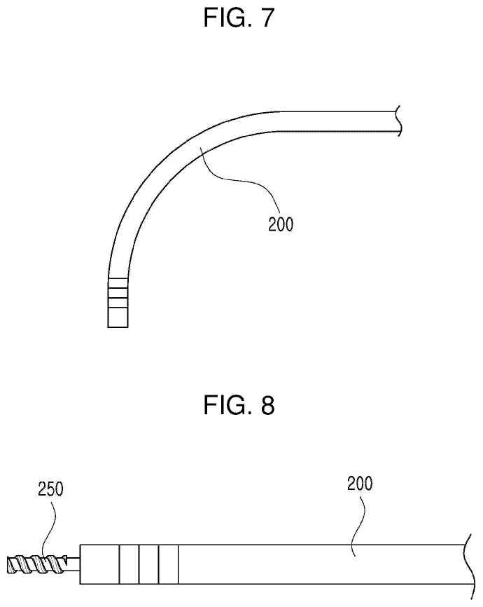

FIG. 1 is an overall schematic diagram of an electrode suction drill system according to an embodiment of the present disclosure, FIGS. 2 and 3 are a side view and a cross-sectional view of a catheter tube according to the present disclosure, FIG. 4 is a view of a drill wire according to the present disclosure, FIG. 5 is an enlarged view of a portion of a manipulator according to the present disclosure, FIGS. 6 and 7 are views of a catheter tube in operation according to the present disclosure, and FIG. 8 is a view of a drill wire advancing and protruding from a catheter tube according to the present disclosure.

One of the main characteristics of the electrode suction drill system according to the present disclosure is the enhanced decompression effect for the treatment of the lumbar herniated intervertebral discs, which is resulted from a combination of the treatment utilizing an electrode and a configuration capable of discharging the lesion tissue to outside. Hereinafter, the present disclosure will be described in detail with reference to the drawings.

Referring to FIG. 1, the electrode suction drill system according to the present disclosure includes a manipulator 100 and a catheter tube 200 attached to a front of the manipulator. For convenience of explanation, “front” means a front side of the manipulator, i.e., the left-hand side as seen in the view of FIG. 1, and “rear” means a rear side of the manipulator, i.e., the right-hand side as seen in the view of FIG. 1. In addition, a vertical direction refers to an up-and-down direction as seen in the view of FIG. 1.

The catheter tube 200 is inserted into the manipulator 100 and coupled therein, and there is a coupling portion 170 provided in the manipulator 100, into which an outer circumferential surface of the catheter tube is inserted so that the catheter tube is fixed at a specific position within the manipulator. The coupling part is fitted with the outer circumference of the catheter tube so that the catheter tube is limited in its movement in a front-and-rear direction with respect to the manipulator.

A first electrode 210 and a second electrode 220 are disposed at a predetermined distance apart from each other at a front end of the catheter tube 200. In addition, it can be seen that a wire (not shown) for supplying the power to the first and second electrodes is provided inside the catheter tube. The processes similar to those of the related art, such as inserting the first and second electrodes into the internal tissues of the intervertebral disc and removing the tissue around the electrodes into a gaseous state by separating the constituent materials of the nucleus pulposus into negatively charged electrons and positively charged ions with a high-energy electric field formed using the radiation frequency, will not be described in detail.

According to the present disclosure, a steering ring 230 is provided at an inner front end of the catheter tube, and first and second wires 231 and 232 are provided to pull the steering ring. That is, one ends of the first and second wires 231 and 232 are coupled to the steering ring, and the other ends of the first and second wires 231 and 232 are coupled to a circular rotating plate 120 inside the manipulator, and an angle adjusting unit 130 is provided on a lower side of the circular rotating plate. When a user places his or her finger in the angle adjusting unit 130 and pulls the same, the circular rotating plate 120 is rotated, and any one of the first and second wires 231 and 232 attached to the circular rotating plate at upper or lower position is pulled. Then, a portion of the steering ring 230 that is attached to the first wire 231 or the second wire 232 being pulled is pulled so that the steering ring is rotated and the tip of the catheter tube is bent due to the tensile force. FIG. 7 illustrates that the tip of the catheter tube is bent by the above operation of the steering ring 130 (hereinafter, referred to as “steering operation” for convenience).

According to the present disclosure, a drill wire 250 is disposed in the inner hollow portion of the catheter tube. The drill wire is connected to a motor M inside the manipulator, such that it may be rotated with respect to a longitudinal direction by receiving the rotational force of the motor. To this end, the motor M, to which one end of the drill wire is fastened, is positioned inside the manipulator. Electric power for rotating the motor is supplied by a battery positioned inside a handle of the manipulator, and a switch 150 for turning on/off the motor operation is positioned on an upper end of the manipulator.

According to the present disclosure, a thread is formed on an outer circumferential surface of the drill wire 150, and the thread includes a tip-side thread 253 formed at a foremost end of the drill wire, and one or more spaced apart threads 255 formed on the outer circumferential surface of the drill wire.

The present disclosure is characterized in that, as the drill wire is rotated, the lesion tissue at the tip of the catheter tube can be suctioned into the inner hollow portion of the catheter tube 200 and then discharged to outside.

Referring to FIG. 3, the catheter tube has an inner diameter of approximately 1.1 mm, and the drill wire has an outer diameter of approximately 1 mm. The thread 253 formed on the outer circumferential surface of the drill wire is positioned between these two diameters, while occupying a space between the inner diameter of the catheter tube and the outer diameter of the drill wire, and the gelatin or the lesion tissue within the disc is suctioned and transported into the catheter tube by the rotation of the thread. While the thread may be formed on the entire outer circumferential surface of the drill wire 150, it may be in the form of a plurality of threads spaced apart at a predetermined interval.

In addition, according to the present disclosure, a motor position adjusting unit 140 is provided on an upper surface of the manipulator 100 to move the motor in the front-and-rear direction. By moving the motor position adjusting unit in the front-and-rear direction (left-and-right direction in FIG. 1), the motor M inside the manipulator is moved by a predetermined distance in the front-and-rear direction. To this end, the motor is mounted to be movable inside the manipulator. That is, a protrusion may be formed on an outer surface of the motor and a groove may be formed inside the manipulator, such that the protrusion may be provided to be movable in the front-and-rear direction while being inserted in the groove.

As described above, since the drill wire of the present disclosure is connected to the motor M inside the manipulator, the drill wire 250 is also moved in the front-and-rear direction according to the movement of the motor in the front-and-rear direction. Meanwhile, the catheter tube is coupled to the coupling part 170 inside the manipulator and fixed in position. Therefore, when the motor is moved in the front-and-rear direction by the motor position adjusting unit, only the drill wire is relatively movable in the front-and-rear direction while the catheter tube remains fixed to the manipulator. That is, the drill wire may be completely inserted into the catheter tube (FIG. 6), or a certain portion of the tip of the drill wire may protrude to the front of the catheter tube (FIG. 8).

According to the present disclosure, the tip of the drill wire is moved relatively in the front-and-rear direction with respect to the catheter tube, in consideration of the steering operation that bends the tip of the catheter tube using the steering ring 230 and the electrode function that uses the first and second electrodes.

That is, when the steering wheel operation is performed with the tip of the drill wire 250 remaining in the protruding state, the steering operation would be insufficient and it would not be smooth, thus resulting in inefficient electrode function. Therefore, when the steering operation is performed, the drill wire is completely inserted into the catheter tube.

The drill wire 250 is 1 mm, which is a relatively thicker wire compared to the first and second wires 231 and 232 pulling the steering ring 230 which are 0.18 mm, and accordingly, if the steering operation is performed while the drill wire remains protruding from the front, the bending motion may not be smooth. In consideration of the above, the steering operation is performed after moving the drill wire backward so as to ensure that the catheter tube is bent enough, and to this end, the motor M is moved in the front-and-rear direction to thus move the drill wire in the front-and-rear direction.

In addition, referring to FIG. 1 again, the present disclosure further includes a discharge member 300 provided inside the manipulator and having an end of the catheter tube inserted therein, and a discharge passage 310 connected with one end to a lower end of the discharge member. The lesion tissue conveyed through the inner portion of the catheter tube is discharged to outside through the discharge member and discharge passage. In order to discharge the lesion tissue to outside, a suction port 320 connected to the other end of the discharge passage is provided. To this end, the material transferred into the catheter tube is discharged outside the manipulator. The drill wire 250 is passed through the discharge member and connected to the motor M.

According to the present disclosure, the catheter is used in the treatment of the lumbar herniated intervertebral discs and the decompression is performed more effectively, since the lesion tissue can be removed concurrently while the treatment using the electrode is performed.

What is claimed is:

1. An electrode suction drill system comprising a manipulator, and a hollow catheter tube mounted on the manipulator, and comprising:
a first electrode and a second electrode provided apart from each other on a front end of the catheter tube;
a steering ring provided at an inner front end of the catheter tube;
first and second wires positioned inside the catheter tube and having one ends connected to the steering ring;
a drill wire provided in an inner hollow portion of the catheter tube;
a thread formed on an outer circumferential surface of the drill wire; and
a motor provided inside the manipulator and fastened to one end of the drill wire to rotate the drill wire, wherein
a tip of the catheter tube performs a steering operation by a manipulation of the first and second wires, and
as the drill wire is rotated by the rotation of the motor, lesion site tissue in a body is suctioned into the catheter tube by the thread.

2. The electrode suction drill system of claim 1, wherein the thread comprises a tip-side thread formed on a foremost end of the drill wire.

3. The electrode suction drill system of claim 2, wherein the thread further comprises one or more spaced apart threads formed on an outer circumferential surface of the drill wire.

4. The electrode suction drill system of claim 1, further comprising a motor position adjusting unit positioned on an upper surface of the manipulator to move the motor in a front-and-rear direction.

5. The electrode suction drill system of claim 4, further comprising a coupling part to fixedly couple the catheter tube inside the manipulator, wherein,
when the motor is moved in the front-and-rear direction by the motor position adjusting unit, the drill wire is moved relatively in the front-and-rear direction while the catheter tube remains fixed to the manipulator.

6. The electrode suction drill system of claim 1, wherein the manipulator further comprises:
a circular rotating plate to pull the first wire or the second wire to thus pull the steering ring, such that the tip of the catheter tube performs the steering operation; and
an angle adjusting unit formed integrally with the circular rotating plate to rotate the circular rotating plate.

7. The electrode suction drill system of claim 1, further comprising:
a discharge member provided inside the manipulator and into which an end of the catheter tube is inserted;
a discharge passage provided inside the manipulator, wherein one end of the discharge passage is connected with the discharge member; and
a suction port provided outside the manipulator and connected to the other end of the discharge passage to discharge the lesion site tissue to outside.
